(19) United States
(12) Patent Application Publication
(10) Pub. No.: US 2025/0367230 A1
(43) Pub. Date: Dec. 4, 2025

(54) Method for preparing SAMe microcapsules

(71) Applicants: XIAMEN KINGDOMWAY BIOTECH. CO., LTD., Fujian (CN); XIAMEN KINGDOMWAY GROUP COMPANY, Fujian (CN)

(72) Inventors: Xuerui Wang, Fujian (CN); Jianhua Lan, Fujian (CN); Jinhong Liu, Fujian (CN); Murong Lin, Fujian (CN); Wenji Wang, Fujian (CN)

(21) Appl. No.: 18/923,558

(22) Filed: Oct. 22, 2024

(30) Foreign Application Priority Data
May 28, 2024 (CN) 202410674385.3

(51) Int. Cl. A61K 31/7076; A61K 9/48; A61K 9/50

(52) U.S. Cl. CPC A61K 31/7076; A61K 9/4866; A61K 9/501; A61K 9/5015; A61K 9/5036; A61K 9/5052; A61K 9/5073; A61K 9/5089

(57) ABSTRACT

The present invention belongs to the field of food or medicine, and relates to a method for preparing SAMe microcapsules. The SAMe microcapsules prepared by the preparation method of the present invention have good stability and anti-hygroscopicity, and are particularly friendly to the gastrointestinal tract.

### TECHNICAL FIELD

The present invention belongs to the field of food or medicine, and relates to a method for preparing SAMe microcapsules.

### BACKGROUND ART

Adenosylmethionine, also known as S-adenosylmethionine (SAM), is a metabolite of the combination of the sulfur-containing amino acid, methionine, and the main energy substance of the human body, adenosine triphosphate (ATP-adenosinetriphosphate). It is a physiologically active substance present in all tissues and body fluids of the human body, and participates in more than 40 biochemical reactions of the body, mainly playing a role in transmethylation, polyamine synthesis and transsulfurization. It has good therapeutic effects on arthritis, depression, liver dysfunction, pancreatitis, etc. In 1999, the US FDA approved S-adenosylmethionine as a health product for listing, and it quickly became one of the best-selling nutritional products in the United States. With the improvement of people's quality of life and the renewal of health concepts, the demand for S-adenosylmethionine will also increase.

S-Adenosyl-L-methionine Disulfate Tosylate (SAMe) is a stable salt of SAM. However, S-adenosylmethionine and its salts are very easy to absorb moisture. After moisture absorption, the SAMe content gradually decreases, which brings great difficulties to the production, storage, and circulation of its preparations, limiting its application in the fields of medical and health care.

At present, there have been studies on chemical modification of S-adenosylmethionine or SAMe to overcome the problem about its hygroscopicity, but the process is relatively complicated and not green or safe. There are also methods that use a higher proportion (more than 40%) of silicon dioxide or calcium hydrogen phosphate to adsorb active ingredient, but this has great defects: the powders prepared by these methods have poor dispersibility, which is not conducive to the use of the formula; the content of ignition residue is high and the bioavailability is low, which is not conducive to human absorption and utilization; at the same time, silicon dioxide or calcium hydrogen phosphate cannot be absorbed or metabolized by the human body, which is not in line with the concept of health.

At present, it is still necessary to develop a new SAMe preparation, especially a preparation process for SAMe microcapsules.

### Contents of the Invention

After in-depth research and creative working, the inventors have obtained a method for preparing SAMe microcapsules. The inventors surprisingly found that the SAMe microcapsules prepared by the preparation method have good stability and anti-hygroscopicity, and are friendly to the gastrointestinal tract. The following invention is therefore provided:

One aspect of the present invention relates to a method for preparing SAMe microcapsules, comprising the following steps:
(1) preparing a core material dispersion and a wall material dispersion;
(2) mixing the core material dispersion and the wall material dispersion evenly to obtain a mixed dispersion;
(3) granulating and drying the mixed dispersion to obtain microcapsules;
wherein,
the core material dispersion comprises SAMe and an acid-resistant filler;
the wall material dispersion is prepared by a method comprising the following steps:
(A) dissolving a protein wall material in water and adjusting the pH value to above 10 to obtain an alkaline protein dispersion;
(B) adding a prebiotic sugar and heating at a temperature of 80° C. to 100° C. for at least 10 minutes.

In step (A), a strong base such as NaOH can be used to adjust the pH to above 10.

In some embodiments of the present invention, in the preparation method, wherein, the core material dispersion is composed of SAMe, the acid-resistant filler and a suitable solvent.

In some embodiments of the present invention, in the preparation method, wherein, the core material dispersion is composed of SAMe, optional cyclodextrin, the acid-resistant filler and a suitable solvent.

In some embodiments of the present invention, in the preparation method, wherein, the wall material dispersion is composed of the protein wall material, the prebiotic sugar and a suitable solvent.

In some embodiments of the present invention, in the preparation method, wherein,
the acid-resistant filler is one or more selected from the group consisting of xanthan gum, sodium alginate, hydroxypropyl methylcellulose, hydroxypropyl cellulose, methylcellulose, chitosan, carboxymethyl cellulose and carboxymethyl starch;
the protein wall material is one or more selected from the group consisting of bovine serum albumin, whey protein, maize zein, casein and soybean protein; and/or
the prebiotic sugar is one or more selected from the group consisting of fructo-oligosaccharide, galacto-oligosaccharide, xylo-oligosaccharide, isomalto-oligosaccharide, soybean oligosaccharide and inulin. Optionally, the polymerization degree of fructo-oligosaccharide, galacto-oligosaccharide, xylo-oligosaccharide, isomalto-oligosaccharide, soybean oligosaccharide is independently 2 to 9, such as 2, 3, 4, 5, 6, 7, 8 or 9. Optionally, the polymerization degree of inulin is 2 to 60, such as 5 to 50, 10 to 45, 10 to 40, 15 to 40, 20 to 35, or 30 to 40.

Prebiotic sugars can stimulate the growth and activity of certain microorganisms in the gastrointestinal tract, be friendly to the gastrointestinal tract, promote the growth and reproduction of beneficial intestinal bacteria, and improve intestinal microecology.

In some embodiments of the present invention, in the preparation method, wherein,
the protein wall material is bovine serum albumin; and
the prebiotic sugar is one or more selected from the group consisting of fructo-oligosaccharide, galacto-oligosaccharide, xylo-oligosaccharide, isomalto-oligosaccharide, soybean oligosaccharide and inulin.

In some embodiments of the present invention, in the preparation method, wherein,
the protein wall material is whey protein; and
the prebiotic sugar is one or more selected from the group consisting of fructo-oligosaccharide, galacto-oligosaccharide, xylo-oligosaccharide, isomalto-oligosaccharide, soybean oligosaccharide and inulin.

In some embodiments of the present invention, in the preparation method, wherein,
the protein wall material is maize zein; and
the prebiotic sugar is one or more selected from the group consisting of fructo-oligosaccharide, galacto-oligosaccharide, xylo-oligosaccharide, isomalto-oligosaccharide, soybean oligosaccharide and inulin.

In some embodiments of the present invention, in the preparation method, wherein,
the protein wall material is casein; and
the prebiotic sugar is one or more selected from the group consisting of fructo-oligosaccharide, galacto-oligosaccharide, xylo-oligosaccharide, isomalto-oligosaccharide, soybean oligosaccharide and inulin.

In some embodiments of the present invention, in the preparation method, wherein,
the protein wall material is soybean protein; and
the prebiotic sugar is one or more selected from the group consisting of fructo-oligosaccharide, galacto-oligosaccharide, xylo-oligosaccharide, isomalto-oligosaccharide, soybean oligosaccharide and inulin.

In some embodiments of the present invention, in the preparation method, wherein,
the SAMe accounts for 50 to 90 parts by weight,
the acid-resistant filler accounts for 0.5 to 5 parts by weight,
the protein wall material accounts for 5 to 12 parts by weight, and/or
the prebiotic sugar accounts for 0.5 to 6 parts by weight.

In some embodiments of the present invention, in the preparation method, wherein,
the SAMe accounts for 50 to 90 parts by weight,
the acid-resistant filler accounts for 0.5 to 5 parts by weight,
the protein wall material accounts for 5 to 10 parts by weight, and/or
the prebiotic sugar accounts for 0.5 to 5 parts by weight.

In some embodiments of the present invention, in the preparation method, wherein,
the SAMe accounts for 60 to 70 parts by weight,
the acid-resistant filler accounts for 1 to 3 or 1 to 2 parts by weight,
the protein wall material accounts for 5 to 10 parts by weight, and/or
the prebiotic sugar accounts for 2 to 5 parts by weight.

In some embodiments of the present invention, in the preparation method, wherein, the core material dispersion further comprises cyclodextrin,
preferably, the cyclodextrin is one or more selected from the group consisting of α-cyclodextrin, β-cyclodextrin and γ-cyclodextrin;
preferably, the cyclodextrin accounts for 1 to 20 parts by weight, 5 to 20 parts by weight, 10 to 20 parts by weight, or 15 to 20 parts by weight.

In the present invention, the cyclodextrin in the SAMe microcapsule not only plays an embedding role, but also plays a synergistic role in the anti-hygroscopicity of the SAMe microcapsule. First, SAMe is highly dispersed in cyclodextrin by grinding. Under the effect of the co-dispersion of cyclodextrin, cyclodextrin changes the aggregation or crystalline form of SAMe, which can improve the hygroscopicity of SAMe.

The present invention uses embedding formula technology to improve the stability of SAMe and reduce its hygroscopicity without changing the structure of SAMe. The preparation process is safe and green, meeting the natural, purity and non-irritating needs pursued by consumers.

In some embodiments of the present invention, in the preparation method, wherein, the core material dispersion is prepared by a method comprising the following steps:
I. grinding SAMe and optional cyclodextrin, passing through an 80 to 120 mesh sieve;
II. dispersing the sieved material in water;
III. adding the acid-resistant filler and mixing them well.

In some embodiments of the present invention, in the preparation method, wherein,
in step I, the grinding is performed using a ball mill, in which the rotation speed is 100 to 350 r/min, and the grinding time is 15 to 30 min;
in step II, the temperature at which the sieved material is dispersed in water is 20° C. to 40° C.; and/or
in step III, the mixing well is achieved by stirring.

In some embodiments of the present invention, the preparation method, wherein,

In step (A), the pH value is 10-12, 11-12 or 10-11.

In some embodiments of the present invention, in the preparation method, wherein,
in step (B), the temperature is 80° C. to 95° C., 80° C. to 90° C., 80° C. to 85° C., 85° C. to 95° C. or 85° C. to 90° C.

In some embodiments of the present invention, in the preparation method, wherein,
in step (B), the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes, or 25 to 30 minutes.

In some embodiments of the present invention, in the preparation method, wherein,
in step (B), the temperature is 80° C. to 95° C., and the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

In some embodiments of the present invention, in the preparation method, wherein,
in step (B), the temperature is 80° C. to 90° C., and
the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

In some embodiments of the present invention, in the preparation method, wherein,
in step (B), the temperature is 80° C. to 85° C., and
the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

In some embodiments of the present invention, in the preparation method, wherein,
in step (B), the temperature is 85° C. to 95° C., and
the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

In some embodiments of the present invention, in the preparation method, wherein,
in step (B), the temperature is 85° C. to 90° C., and
the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

In some embodiments of the present invention, in the preparation method, wherein,
in step (B), the stirring is performed while heating.

In some embodiments of the present invention, in the preparation method, wherein,
in step (2), the core material dispersion and the wall material dispersion are homogenously mixed by stirring or ultrasonic treatment;
preferably, the ultrasonic treatment is performed under negative pressure;
preferably, the ultrasonic treatment conditions are as follows: pressure is −0.07 MPa to −0.1 MPa, ultrasonic power is 240 to 480 w (preferably 300 to 400 w), time is 6 to 8 minutes.

In some embodiments of the present invention, in the preparation method, wherein, the mixed dispersion in step (2) is stored at 20° C. to 40° C.

In some embodiments of the present invention, in the preparation method, wherein,
in step (3), a microcapsule of 40 to 70 mesh is obtained.

The particle size of SAMe microcapsule is controlled within a suitable range (powder particle size is between 40 and 70 mesh) to optimize the compressibility of the particles, and the powder can be directly compressed into tablets without adding other excipients, and the smaller the pressure used to achieve the required hardness (usually more than 5 kg) during tablet compression, the smaller the influence of mechanical damage (instantaneous punching) on the active substance.

Technical means known to those skilled in the art can be used to adjust or control the particle size or diameter of the microcapsules, such as by adjusting the atomizer frequency, the viscosity of the mixed dispersion and/or the feed flow rate. Optionally, the microcapsules of 40 to 70 mesh are obtained by sieving. The particle size corresponding to 40 mesh is 425 microns, and the particle size corresponding to 70 mesh is 212 microns.

In some embodiments of the present invention, in the preparation method, wherein,
the mixed dispersion is granulated and dried by spray drying to obtain microcapsules;
preferably, the mixed dispersion is granulated and dried by spray fluidized-bed drying to obtain microcapsules.

In some embodiments of the present invention, in the preparation method, wherein,
in step (3), silicon dioxide and corn starch are evenly mixed as a coating material, and the mixed dispersion obtained in step (2) is sprayed and fluidized-bed dried in a spray dryer to obtain microcapsules.

In some embodiments of the present invention, in the preparation method, wherein,
in step (3), the silicon dioxide accounts for 0.1 to 1 parts by weight, and/or the corn starch accounts for 5 to 25 parts by weight.

In some embodiments of the present invention, in the preparation method, wherein, in step (3),
the spray tower inlet air temperature is controlled at 140° C. to 160° C., the fluidized-bed temperature is controlled at 40° C. to 45° C., and the atomizer frequency is 24 to 26 Hz.

Corn starch is used as the coating wall material to prepare three-layer microcapsules through a fluidized bed-spray drying process. This coating layer can effectively improve the embedding effect of the microcapsules, not only to reinforce the microcapsule shell, but also to fill the cracks and holes that may appear in the capsule wall during the spray drying process, so that the embedding effect of the microcapsules is further enhanced, thereby obtaining high-quality SAMe microcapsule products and effectively reducing hygroscopicity.

Without being limited by any theory, the first layer of the membrane is formed by the acid-resistant filler, for example, the cyclodextrin cavity can embed SAMe by and the gel structure of xanthan gum can provide protective effect on SAMe; the second layer of the membrane is formed by the wall material dispersion; and the third layer is formed by starch (e.g., corn starch). All three layers of the membrane are solidified and formed during the spray drying process.

The three-layer membrane structure of the microcapsule of the present invention better isolates moisture, stabilizes product quality, effectively reduces hygroscopicity, and the powder has good pressure resistance and fluidity, and can be directly used to prepare tablet products.

In some embodiments of the present invention, the preparation method comprises the following steps:
1) SAMe is mixed with an appropriate amount of cyclodextrin, placed in a ball mill, ground at a speed of 100 to 350 r/min for 15 to 30 minutes, and passed through an 80 to 120 mesh sieve;
2) the product of step 1) is dispersed in purified water at 20° C. to 40° C.;
3) an appropriate amount of an acid-resistant filler is added and mixed well;
4) an appropriate amount of a protein wall material is dissolved in water, adjusted to have a pH value of 10 to 12, thereby obtaining an alkaline protein aqueous solution, and then an appropriate amount of the prebiotic sugar is added, heated to 80° C. to 95° C., stirred for 15 to 30 minutes, and reacted under thorough stirring;
5) the product of step 3) is mixed with the product of step 4), then subjected to ultrasonic treatment under negative pressure (the pressure is −0.07 MPa to −0.1 MPa) with an ultrasonic power of 240 to 480 w (preferably 300 to 400 w) and a treatment time of 6 to 8 minutes to obtain a uniform mixed dispersion, and a temperature kept at 20° C. to 40° C.;
6) an appropriate amount of silicon dioxide is added to corn starch and mixed well, and used as a coating material to perform spray fluidized-bed drying of the mixed dispersion of step 5) in a spray dryer with a spray tower inlet temperature controlled at 140° C. to 160° C., a fluidized-bed temperature controlled at 40° C. to 45° C., and an atomizer frequency at 24 to 26 Hz; after being sieved, a SAMe microcapsule with a particle size between 40 and 70 mesh is obtained.

In some embodiments of the present invention, the preparation method comprises the following steps:
i) 50 to 90 parts by weight of SAMe is mixed with an appropriate amount of cyclodextrin, placed in a ball mill, ground at a speed of 100 to 350 r/min for 15 to 30 minutes, and passed through an 80 to 120 mesh sieve;
ii) the product of step i) is dispersed in purified water at 20° C. to 40° C.;
iii) 0.5 to 5 parts by weight of an acid-resistant filler is added and mixed well;
iv) 5 to 10 parts by weight of a protein wall material is dissolved in water, adjusted to have a pH value of 10 to 12, thereby obtaining an alkaline protein aqueous solution, and then 0.5 to 5 parts by weight of a prebiotic sugar is added, heated to 80 to 95° C., stirred for 15 to 30 minutes, and reacted under thorough stirring;
v) the product of step iii) and the product of step iv) are mixed, then subjected to ultrasonic treatment under negative pressure (the pressure is −0.07 MPa to −0.1 MPa) with an ultrasonic power of 240 to 480 w (preferably 300 to 400 w) and a treatment time of 6 to 8 minutes to obtain a uniform mixed dispersion, and a temperature kept at 20° C. to 40° C.;
vi) an appropriate amount of silicon dioxide is added to corn starch and mixed well, and used as a coating material to perform spray fluidized-bed drying of the mixed dispersion of step v) in a spray dryer with a spray tower inlet temperature controlled at 140° C. to 160° C., a fluidized-bed temperature controlled at 40° C. 45° C., and an atomizer frequency at 24 to 26 Hz; after being sieved, a SAMe microcapsule with a particle size between 40 and 70 mesh is obtained.

Another aspect of the present invention relates to a SAMe microcapsule, which is prepared by the preparation method of any one of items described in the present invention.

Another aspect of the present invention relates to a composition, which comprises the SAMe microcapsule of the present invention and one or more pharmaceutically or bromatologically acceptable excipients;
preferably, the composition is a tablet, soft capsule, hard capsule, powder, or pill.

In the present invention, unless otherwise specified, the “core material dispersion” refers to a dispersion containing the core material, that is, besides containing the core material (SAMe), it is not excluded that other components other than the core material, such as acid-resistant filler and cyclodextrin, may also be contained. In some cases, the acid-resistant filler and cyclodextrin act as wall materials.

In the present invention, unless otherwise specified, the “wall material dispersion” does not contain the core material.

### Beneficial Effects of the Invention

The present invention achieves one or more of the following technical effects (1) to (4):
(1) the prepared microcapsules have good anti-hygroscopicity and stability;
(2) they are more friendly to the gastrointestinal tract and can reduce the acidity of the system. The present invention effectively alleviates the technical problem that long-term use will damage the gastrointestinal system, cause gastrointestinal dysbiosis, and cause gastrointestinal reactions;
(3) the preparation process and subsequent tableting process of SAMe microcapsules produced by the method of the present invention do not require strict moisture control in the production environment, and the humidity requirement for the production environment is not high. The initial moisture content is low, the ignition residue content is low, and the storage time at room temperature is long;
(4) the SAMe microcapsule powder prepared by the present invention has good compressibility, and the particle shape is a solid and compact spherical structure with a smooth and regular surface.

### Specific Models for Carrying Out the Invention

The following will describe the embodiments of the present invention in detail in conjunction with the examples, but those skilled in the art will understand that the following examples are only used to illustrate the present invention and should not be regarded as limiting the scope of the present invention. If the specific conditions were not specified in the examples, they were carried out according to conventional conditions or the conditions recommended by the manufacturer. The reagents or instruments used without indicating the manufacturer were all conventional products that could be purchased commercially.

As used in the examples, α-cyclodextrin had a molecular weight of is 972.84, β-cyclodextrin had a molecular weight of 1134.98, and 7-cyclodextrin had a molecular weight of 1297.12, and all of which were purchased from Zibo Qianhui Biotechnology Co., Ltd. The soybean protein used in the examples had model number of GS5300D, and was purchased from Gushen Biotechnology Group Co., Ltd.

The coating ratio referred to the percentage of starch weight to the total weight of the microcapsule.

The detection method of SAMe content in microcapsules referred to the detection method of S-Adenosyl-L-methionine Disulfate Tosylate in the United States Pharmacopoeia.

### Example 1

1. 65 g of SAMe and 18 g of β-cyclodextrin were mixed, placed in a ball mill, ground at a speed of 200 r/min for 20 min, and passed through a 100-mesh sieve.
2. The product of step 1 was dispersed in 60 g of purified water at 40° C.
3. 1 g of xanthan gum and 0.5 g of HPMC were added, stirred and mixed well during the addition process.
4. 10 g of soybean protein was dissolved in 20 g of water, adjusted with liquid caustic soda to reach a pH value of 11 to obtain an alkaline protein aqueous solution, then added with 5 g of isomalto-oligosaccharide (polymerization degree was 5, the same below), heated to 90° C., and stirred thoroughly for 25 minutes.
5. The product of step 3 and the product of step 4 were mixed, and then subjected to ultrasonic treatment under negative pressure (−0.08 Mpa) with an ultrasonic power of 300 w and an ultrasonic treatment time of 6 minutes to obtain a uniform mixed dispersion, and a temperature kept at 40° C.
6. 0.5 g of silicon dioxide was added to corn starch and mixed well to form a coating material, which was used to perform spray fluidized-bed drying of the mixed dispersion obtained in step 5 in a spray dryer with a spray tower inlet temperature controlled at 148° C., a fluidized-bed temperature controlled at 45° C., and an atomizer frequency at 25 Hz. After being sieved, SAMe microcapsules with a particle size between 40 and 70 mesh were finally obtained. After testing, the coating ratio of corn starch was 20%, and the content of SAMe in the microcapsules was 52.6%.

### Example 2

1. 83 g of SAMe and 8 g of β-cyclodextrin were mixed, placed in a ball mill, ground at a speed of 350 r/min for 15 minutes, and passed through a 100-mesh sieve.
2. The product of step 1 was dispersed in 50 g of purified water at 40° C.
3. 0.5 g of sodium alginate and 0.5 g of xanthan gum were added, stirred and mixed during the addition process.
4. 6 g of whey protein was dissolved in 10 g of water, adjusted to reach a pH value of 12 with liquid caustic soda to obtain an alkaline protein aqueous solution, and then added with 2 g of fructo-oligosaccharide (polymerization degree was 4, the same below), heated to 85° C., and stirred thoroughly for 30 minutes.
5. The product of step 3 and the product of step 4 were mixed, and then subjected to ultrasonic treatment under negative pressure (−0.07 Mpa) with an ultrasonic power of 350 w and an ultrasonic treatment time of 7 minutes to obtain a uniform mixed dispersion, and a temperature kept at 40° C.
6. 0.5 g of silicon dioxide was added to corn starch and mixed well to form a coating material, which was used to perform spray fluidized-bed drying of the mixed dispersion obtained in step 5 in a spray dryer with a spray tower inlet temperature controlled at 155° C., a fluidized-bed temperature controlled at 45° C., and an atomizer frequency at 24.5 Hz. After being sieved, SAMe microcapsules with a particle size between 40 and 70 mesh were finally obtained. After testing, the coating ratio of corn starch was 15%, and the SAMe content in the microcapsules was 70.3%.

### Example 3

1. 80 g of SAMe and 10 g of 7-cyclodextrin were mixed, placed in a ball mill, ground at a speed of 250 r/min for 25 minutes, and passed through an 80-120 mesh sieve.
2. The product of step 1 was dispersed in 60 g of purified water at 35° C.
3. 2 g xanthan gum was added, stirred and mixed during the addition process.
4. 5 g of casein was dissolved in 15 g of water, adjusted to reach a pH of 11 with liquid caustic soda to obtain an alkaline protein aqueous solution, then added with 3 g of isomalto-oligosaccharide, heated to 95° C., and stirred thoroughly for 25 minutes.
5. The product of step 3 and the product of step 4 were mixed, and then subjected to ultrasonic treatment under negative pressure (−0.08 Mpa) with an ultrasonic power of 400 w and an ultrasonic treatment time of 8 minutes to obtain a uniform mixed dispersion, and a temperature kept at 35° C.
6. 0.5 g of silicon dioxide was added to corn starch and mixed well to form a coating material, which was used to perform spray fluidized-bed drying of the mixed dispersion obtained in step 5 in a spray dryer with a spray tower inlet air temperature controlled at 150° C., a fluidized-bed temperature controlled at 40° C., and an atomizer frequency at 26 Hz. After being sieved, SAMe microcapsules with a particle size between 40 and 70 mesh were finally obtained. After testing, the coating ratio of corn starch was 25%, and the SAMe content in the microcapsules was 60.6%.

### Example 4

1. 67 g of SAMe and 17 g of α-cyclodextrin were mixed, placed in a ball mill, ground at a speed of 100 r/min for 20 minutes, and passed through an 80-mesh sieve.
2. The product of step 1 was dispersed in 65 g of purified water at 35° C.
3. 0.5 g of sodium alginate, 0.5 g of methylcellulose and 1 g of HPMC were added, stirred and mixed during the addition process.
4. 10 g of bovine serum albumin was dissolved in 15 g of water, adjusted to reach a pH value of 10 with liquid caustic soda to obtain an alkaline protein aqueous solution, and then added with 4 g of isomalto-oligosaccharide, heated to 95° C., and stirred thoroughly for 30 minutes.
5. The product of step 3 and the product of step 4 were mixed, and then subjected to ultrasonic treatment under negative pressure (−0.07 Mpa) with an ultrasound power of 300 w and an ultrasonic treatment time of 7 min to obtain a uniform mixed dispersion, and a temperature kept at 35° C.
6. 0.5 g of silicon dioxide was added to corn starch and mixed well to form a coating material, which was used to perform spray fluidized-bed drying of the mixed dispersion obtained in step 5 in a spray dryer with a spray tower inlet temperature controlled at 145° C., a fluidized-bed temperature controlled at 40° C., and an atomizer frequency at 25.5 Hz. After being sieved, SAMe microcapsules with a particle size between 40 and 70 mesh were finally obtained. After testing, the coating ratio of corn starch was 25%, and the SAMe content in the microcapsules was 50.7%.

### Example 5

1. 88 g of SAMe and 5 g of β-cyclodextrin were mixed, placed in a ball mill, ground at a speed of 350 r/min for 15 minutes, and passed through a 120-mesh sieve.
2. The product of step 1 was dispersed in 50 g of purified water at 20° C.
3. 1 g of xanthan gum was added, stirred and mixed during the addition process.
4. 5 g of soybean protein was dissolved in 15 g water, adjusted to reach a pH value of 11 with liquid caustic soda to obtain an alkaline protein aqueous solution, then added with 1 g of galacto-oligosaccharide (polymerization degree was 3, the same below), heated to 90° C., and stirred thoroughly for 20 minutes.
5. The product of step 3 and the product of step 4 were mixed, and then subjected to ultrasonic treatment under negative pressure (−0.08 Mpa) with an ultrasonic power of 400 w and an ultrasonic treatment time of 8 minutes to obtain a uniform mixed dispersion, and a temperature kept at 20° C.
6. 0.5 g of silicon dioxide was added to corn starch and mixed well to form a coating material, which was used to perform spray fluidized-bed drying of the mixed dispersion obtained in step 5 in a spray dryer with a spray tower inlet temperature controlled at 158° C., a fluidized-bed temperature controlled at 40° C., and an atomizer frequency at 25 Hz. After being sieved, SAMe microcapsules with a particle size between 40 and 70 meshes were finally obtained. After testing, the coating ratio of corn starch was 20%, and the content of SAMe in the microcapsules was 70.5%.

### Comparative Example 1

The β-cyclodextrin was replaced with porous starch of the same weight, and the rest was the same as the formula and preparation process of Example 1. After testing, the content of SAMe in the microcapsules was 49.8%.

### Comparative Example 2

The same formula and preparation process as those in Example 1 were adopted, except that: SAMe and β-cyclodextrin were simply mixed without grinding. After testing, the content of SAMe in the microcapsules was 48.5%.

### Comparative Example 3

The same formula and preparation process as those in Example 1 were adopted, except that: in step 4, after dissolving soybean protein with 20 g water, 5 g of isomalto-oligosaccharide was added, heated to 90° C., and stirred thoroughly for 25 minutes; in which the protein was not subjected to the treatment with alkali. The rest of the steps were the same as those in Example 1. After testing, the content of SAMe in the microcapsules was 51.8%.

### Comparative Example 4

1. 65 g of SAMe and 33 g of β-cyclodextrin were mixed, placed in a ball mill, ground at a speed of 200 r/min for 20 minutes, and passed through a 100-mesh sieve.
2. The product of step 1 was dispersed in 60 g of purified water at 40° C.
3. 1 g of xanthan gum and 0.5 g of HPMC were added, stirred and mixed well during the addition process.
4. After the mixing, ultrasonic treatment was carried out under negative pressure (−0.08 Mpa) with an ultrasonic power of 300 w and an ultrasonic treatment time of 6 minutes to obtain a uniform mixed dispersion, and a temperature kept at 40° C.
5. 0.5 g of silicon dioxide was added to corn starch and mixed well to form a coating material, which was used to perform spray fluidized-bed drying of the mixed dispersion obtained in step 4 in a spray dryer with a spray tower inlet temperature controlled at 148° C., a fluidized-bed temperature controlled at 45° C., and an atomizer frequency at 25 Hz. After being sieved, SAMe microcapsules with a particle size between 40 and 70 mesh were finally obtained. After testing, the coating ratio of corn starch was 20%, and the content of SAMe in the microcapsule was 52.2%.

### Comparative Example 5

The formula and preparation process were the same as those in Example 2, except that: in step 4, after the whey protein was dissolved in 10 g of water, 2 g of fructo-oligosaccharide was added, heated to 85° C., and stirred thoroughly for 30 minutes; in which the protein was not subjected to the treatment with alkali, and the remaining steps were the same as those in Example 2. After testing, the content of SAMe in the microcapsules was 70.2%.

### COMPARATIVE EXAMPLE 6

The formula and preparation process were the same as those in Example 3, except that: in step 4, after the casein was dissolved in 15 g of water, 3 g of isomalto-oligosaccharide was added, heated to 95° C., and stirred thoroughly for 25 minutes; in which the protein was not subjected to the treatment with alkali, and the remaining steps were the same as those in Example 3. After testing, the content of SAMe in the microcapsules was 59.3%.

### Comparative Example 7

The same formula and preparation process were the same as those in Example 4, except that: in step 4, after dissolving bovine serum albumin with 15 g water, 4 g of isomalto-oligosaccharide was added, heated to 95° C., and stirred thoroughly for 30 minutes; that was, the protein was subjected to the treatment with alkali, and the remaining steps were the same as those in Example 4. After testing, the content of SAMe in the microcapsules was 50.3%.

### Comparative Example 8

The same formula and preparation process were the same as those in Example 5, except that: in step 4, after dissolving soybean protein with 15 g water, 1 g of galacto-oligosaccharide was added, heated to 90° C., and stirred thoroughly for 20 minutes; the protein was not subjected to the treatment with alkali, and the remaining steps were the same as those in Example 5. After testing, the content of SAMe in the microcapsules was 70.0%.

### Comparative Example 9

The same formula and preparation process were the same as those in Example 1, except that: in step 4, the pH was adjusted to 13, and the remaining steps were the same as those in Example 1. After testing, the content of SAMe in the microcapsules was 50.6%.

### Test Example 1: Experiment of Hygroscopicity

Samples: SAMe microcapsules prepared in Examples 1 to 5. Reference samples were those of Comparative Examples 1 to 9.

Appropriate amounts of the samples to be tested were accurately weighed, and spread on the bottom of weighing bottle (thickness was less than 5 mm). The moisture absorption of the microcapsules was tested at different time periods at an environment temperature of about 25° C. and an environment humidity of about 60%. The results were shown in Table 1.

TABLE 1

| Sample | 1 h | 2 h | 4 h | 8 h | 12 h | 24 h |
|---|---|---|---|---|---|---|
| Example 1 | 0% | 0% | 0% | 0% | 1% | 1.5% |
| Example 2 | 0% | 0% | 0% | 0.6% | 1.9% | 2.8% |
| Example 3 | 0% | 0% | 0% | 0% | 0.8% | 1.2% |
| Example 4 | 0% | 0% | 0% | 0% | 0.6% | 1.1% |
| Example 5 | 0% | 0% | 0% | 0.5% | 1.6% | 2.7% |
| Comparative Example 1 | 0% | 0% | 0.2% | 1.9% | 4.5% | 7.3% |
| Comparative Example 2 | 0% | 0% | 0% | 0.4% | 2.1% | 4.7% |
| Comparative Example 3 | 0% | 0.1% | 1.4% | 3.1% | 7.5% | 10.5% |
| Comparative Example 4 | 0% | 0.7% | 4.9% | 8.5% | 10.1% | 15.5% |
| Comparative Example 5 | 0% | 0.05% | 0.98% | 2.9% | 7.8% | 11.2% |
| Comparative Example 6 | 0% | 0.04% | 1.03% | 3.2% | 8.1% | 12.3% |
| Comparative Example 7 | 0% | 0.1% | 1.3% | 3.8% | 10.3% | 11.5% |
| Comparative Example 8 | 0% | 0.08% | 1.3% | 3.3% | 8.1% | 11% |
| Comparative Example 9 | 0% | 0.2% | 0.5% | 4.3% | 7.9% | 11.6% |

The results in Table 1 showed that the SAMe microcapsules prepared by the present invention had good anti-hygroscopicity and could remain stable.

### Test Example 2: Experiment of Particle Powder Determination

The powder properties of the granules after granulation included angle of repose, bulk density, and tap density.

The specific test methods were as follows:

Angle of repose: BT 1001 intelligent powder tester was used, an appropriate amount of microcapsules was taken, the angle of repose program was selected, a funnel with an aperture of 5 mm was used, a feed rate of 2 was set, then the feeding was started, and the results were recorded.

Bulk density: BT 1001 intelligent powder tester was used, an appropriate amount of microcapsules was taken, the bulk density program was selected, a funnel with an aperture of 5 mm was used, a feed rate of 2 was set, the test type was set as non-metal, the balance (empty cup) was read, the feeding was started, the balance (full cup) was read after the feeding, and the bulk density results were recorded.

Tap density: BT 1001 intelligent powder tester was used, an appropriate amount of microcapsules was taken, the tap density program was selected for measurement, the tap frequency was set as 100, the tap times was set as 500, the test type was set as fixed volume, the balance (empty cup) was read, the tapping was started after adding material, the balance (full cup) was read after tapping, and the tap density results were recorded.

The results were shown in Table 2.

TABLE 2

| Sample | Angle of repose | Bulk density | Tap density |
|---|---|---|---|
| Example 1 | 19.23 | 0.68 | 0.83 |
| Example 2 | 19.56 | 0.67 | 0.83 |
| Example 3 | 19.02 | 0.69 | 0.86 |
| Example 4 | 19.76 | 0.68 | 0.84 |
| Example 5 | 19.12 | 0.66 | 0.82 |
| Comparative Example 1 | 22.35 | 0.51 | 0.72 |
| Comparative Example 2 | 20.38 | 0.55 | 0.75 |
| Comparative Example 3 | 23.45 | 0.53 | 0.75 |
| Comparative Example 4 | 22.79 | 0.51 | 0.74 |
| Comparative Example 5 | 23.45 | 0.49 | 0.72 |
| Comparative Example 6 | 24 | 0.51 | 0.74 |
| Comparative Example 7 | 26 | 0.52 | 0.74 |
| Comparative Example 8 | 25 | 0.51 | 0.75 |
| Comparative Example 9 | 25.5 | 0.56 | 0.79 |

Those skilled in the art knew that the smaller the angle of repose, the better the fluidity, the more conducive to subsequent processing and utilization, such as packaging, mixing, etc. Because SAMe itself had a high density, the high bulk density and tap density indicated that the content of the active ingredient SAMe was high, which could reduce the intake of non-active ingredients during use. At the same time, the high bulk density and tap density had better effect on tableting, which could improve the compressibility of powder to a certain extent.

### Test Example 3: Experiment of Direct Tabletting

A rotary tabletting machine was used for tabletting, an appropriate punch was selected, appropriate tablet weight and pressure were adjusted, and tabletting was carried out.

The results were shown in Table 3.

TABLE 3

| Sample | Tabletting pressure, KN | Tablet hardness, kg |
|---|---|---|
| Example 1 | 5 | 7 |
| Example 2 | 5 | 8 |
| Example 3 | 5 | 7.5 |
| Example 4 | 5 | 8 |
| Example 5 | 5 | 8.5 |
| Comparative Example 1 | 5 | 5.8 |
| Comparative Example 2 | 5 | 6.6 |
| Comparative Example 3 | 5 | 6.1 |
| Comparative Example 4 | 5 | 5.5 |
| Comparative Example 5 | 5 | 5.4 |
| Comparative Example 6 | 5 | 5.5 |
| Comparative Example 7 | 5 | 5.6 |
| Comparative Example 8 | 5 | 5.9 |
| Comparative Example 9 | 5 | 5.8 |

The SAMe microcapsules prepared by the present invention had good compressibility, and the particle shape was a regular compact spherical structure, so that the particles had good compressibility, and the powder could be directly tabletted without adding other auxiliary materials. When tabletting, a tabletting pressure of about 5 KN could achieve a tablet hardness of 7 kg or more. Since the pressure required for tabletting was small, the mechanical damage (instant punching) had less impact on the active substance. Generally, the particles prepared by the dry method had almost no compressibility, and they relied entirely on the bonding effect between the particles and could only be pressed into tablets with greater pressure.

Because the SAMe microcapsule particles prepared by the present invention had excellent particle fluidity and compressibility, the tablets prepared by the present invention were obtained by directly pressing the SAMe microcapsule particles without adding other auxiliary tabletting materials; the tablet hardness met the requirements, which effectively avoided the problem of reduced purity (content) of SAMe in the tablets caused by adding other auxiliary compressive auxiliary ingredients such as magnesium stearate to increase the hardness of tablets in the prior art.

### Test Example 4: Determination of pH Value of SAMe Microcapsules

The SAMe microcapsules prepared by the formula in Examples 1 to 5 were taken, and the reference samples were those of Comparative Examples 1 to 9. 1 g of each of the above samples was taken and added to 100 g of water, and the pH value was measured.

The results were shown in Table 4.

TABLE 4

| Sample | pH value |
|---|---|
| Example 1 | 1.93 |
| Example 2 | 1.88 |
| Example 3 | 1.90 |
| Example 4 | 1.91 |
| Example 5 | 1.86 |
| Comparative Example 1 | 1.85 |
| Comparative Example 2 | 1.90 |
| Comparative Example 3 | 1.65 |
| Comparative Example 4 | 1.51 |
| Comparative Example 5 | 1.54 |
| Comparative Example 6 | 1.53 |
| Comparative Example 7 | 1.51 |
| Comparative Example 8 | 1.56 |
| Comparative Example 9 | 1.95 |
| SAMe raw material | 1.3 |

The results in Table 4 showed that the microcapsules prepared by the present invention had a relatively high pH value after dispersion, which could reduce the acidity of the dispersion system, thereby helping to protect the gastrointestinal tract.

### Test Example 5: Effect on Mouse Gastrointestinal Tract

Experimental animals: Healthy adult mice (C57, purchased from Pabeile Experimental Animal Breeding Co., Ltd.), with body weight of 18 to 22 g.

Experimental materials: The test samples were SAMe microcapsules obtained from the formulas in Examples 1 to 5 and Comparative Examples 1 to 9, and SAMe raw material.

Experimental method: The designed dose (5.0 g/kg·bw) was equivalent to 5 times the recommended dosage for humans, and an ulcer model control group was set. Each group was gavaged with the test sample once a day, and the control group was fed with an equal volume of distilled water. After 21 consecutive days of feeding, the animals were fasted for 1 day. 0.5 h after the last gavage, the mice of each group were intraperitoneally injected with indomethacin suspension (purchased from Liaoyang Helin Pharmaceutical Co., Ltd.) (10 mg/kg·bw), and 1 h later, the mice were gavaged with 60% ethanol (0.1 mg/kg·bw). After 1 h, the mice were killed by cervical dislocation, laparotomy and ligation of pylorus were carried out, and 1 mL of 1% formalin was orally gavaged. The cardia was immediately ligated and the entire stomach was cut off. After flushing the stomach contents, the stomach was spread on a glass plate. Under a shadowless magnifying lamp, the area of gastric mucosal lesions was measured and statistically analyzed.

The results were shown in Table 5.

TABLE 5

| | Number | Lesion area | |
|---|---|---|---|
| Sample | of mice | (mm²) | P |
| Example 1 | 10 | 5.26 ± 3.03 | P < 0.01 |
| Example 2 | 10 | 5.68 ± 2.17 | P < 0.01 |
| Example 3 | 10 | 5.45 ± 2.25 | P < 0.01 |
| Example 4 | 10 | 5.13 ± 1.98 | P < 0.01 |
| Example 5 | 10 | 5.71 ± 2.31 | P < 0.01 |
| Comparative Example 1 | 10 | 5.83 ± 2.74 | P < 0.01 |
| Comparative Example 2 | 10 | 5.43 ± 2.98 | P < 0.01 |
| Comparative Example 3 | 10 | 7.98 ± 3.56 | P < 0.01 |
| Comparative Example 4 | 10 | 9.69 ± 3.43 | P < 0.01 |
| Comparative Example 5 | 10 | 7.93 ± 3.22 | P < 0.01 |
| Comparative Example 6 | 10 | 7.88 ± 3.55 | P < 0.01 |
| Comparative Example 7 | 10 | 7.84 ± 3.47 | P < 0.01 |
| Comparative Example 8 | 10 | 7.83 ± 3.25 | P < 0.01 |
| Comparative Example 9 | 10 | 7.52 ± 3.02 | P < 0.01 |
| SAMe raw material | 10 | 11.57 ± 4.35 | P < 0.01 |
| Ulcer model group | 10 | 15.83 ± 6.51 | / |

The results showed that ulcer lesions occurred in all groups, and the gastric mucosal lesion area of the mice of each dose group was smaller than that of the ulcer model control group, and the differences were highly significant (P<0.01). However, the mucosal lesion areas of Examples 1 to 5 groups were significantly smaller than those of the control groups 3 to 9, the SAMe raw material group and the ulcer model group, and the differences were highly significant, indicating that the SAMe microcapsules prepared by the present invention had a certain protective effect on the gastric wall.

Although the specific models of the present invention have been described in detail, it will be understood by those skilled in the art that various modifications and substitutions may be made to those details according to all the teachings disclosed, and such changes are within the scope of protection of the present invention. The full scope of the present invention is given by the appended claims and any equivalents thereof.

What is claimed is:

1. A method for preparing SAMe microcapsules, comprising the following steps:
(1) preparing a core material dispersion and a wall material dispersion;
(2) mixing well the core material dispersion and the wall material dispersion to obtain a mixed dispersion;
(3) granulating and drying the mixed dispersion to obtain microcapsules;
wherein,
the core material dispersion comprises SAMe and an acid-resistant filler;
the wall material dispersion is prepared by a method comprising the following steps:
(A) dissolving a protein wall material in water, and adjusting the pH value to above 10 to obtain an alkaline protein dispersion;
(B) adding a prebiotic sugar, and heating at a temperature of 80° C. to 100° C. for at least 10 minutes.

2. The preparation method according to claim 1, wherein,
the acid-resistant filler is one or more selected from the group consisting of xanthan gum, sodium alginate, hydroxypropyl methylcellulose, hydroxypropyl cellulose, methylcellulose, chitosan, carboxymethyl cellulose and carboxymethyl starch;
the protein wall material is one or more selected from the group consisting of bovine serum albumin, whey protein, maize zein, casein and soybean protein; and/or
the prebiotic sugar is one or more selected from the group consisting of fructo-oligosaccharide, galacto-oligosaccharide, xylo-oligosaccharide, isomalto-oligosaccharide, soybean oligosaccharide and inulin.

3. The preparation method according to claim 1, wherein,
the SAMe accounts for 50 to 90 parts by weight,
the acid-resistant filler accounts for 0.5 to 5 parts by weight,
the protein wall material accounts for 5 to 12 parts by weight, and/or
the prebiotic sugar accounts for 0.5 to 6 parts by weight.

4. The preparation method according to claim 1, wherein the core material dispersion further comprises cyclodextrin,
preferably, the cyclodextrin is one or more selected from the group consisting of α-cyclodextrin, β-cyclodextrin and 7-cyclodextrin;
preferably, the cyclodextrin accounts for 1 to 20 parts by weight.

5. The preparation method according to claim 1, wherein the core material dispersion is prepared by a method comprising the following steps:
I. grinding SAMe and optional cyclodextrin, passing through an 80 to 120 mesh sieve;
II. dispersing the sieved material in water;
III. adding the acid-resistant filler and mixing them well.

6. The preparation method according to claim 5, wherein:
in step I, the grinding is performed using a ball mill with a rotation speed of 100 to 350 r/min and a grinding time of 15 to 30 minutes;
in step II, the temperature at which the sieved material is dispersed in water is 20° C. to 40° C.; and/or
in step III, the mixing well is achieved by stirring.

7. The preparation method according to claim 1, wherein, in step (A), the pH value is 10 to 12, 11 to 12 or 10 to 11.

8. The preparation method according to claim 1, wherein, in step (B), the temperature is 80° C. to 95° C., 80° C. to 90° C., 80° C. to 85° C., 85° C. to 95° C. or 85° C. to 90° C.

9. The preparation method according to claim 1, wherein, in step (B), the heating time is 10 to 90 minutes, 15 to 80 minutes, 15 to 70 minutes, 15 to 60 minutes, 15 to 50 minutes, 15 to 40 minutes, 15 to 30 minutes, 15 to 25 minutes, 15 to 20 minutes, 20 to 30 minutes, 20 to 25 minutes or 25 to 30 minutes.

10. The preparation method according to claim 1, wherein, in step (B), the stirring is performed while heating.

11. The preparation method according to claim 1, wherein, in step (2), the core material dispersion and the wall material dispersion are mixed well by stirring or ultrasonic treatment;
preferably, the ultrasonic treatment is an ultrasonic treatment performed under negative pressure;
preferably, the ultrasonic conditions are as follows:
pressure is −0.07 MPa to −0.1 MPa, ultrasonic power is 240 to 480 w (preferably 300 to 400 w), and time is 6 to 8 minutes.

12. The preparation method according to claim 1, wherein, in step (2), the mixed dispersion is stored at 35° C. to 45° C.

13. The preparation method according to claim 1, wherein, in step (3), microcapsules of 40-70 mesh are obtained.

14. The preparation method according to claim 1, wherein, in step (3),
the mixed dispersion is granulated and dried by spray drying to obtain microcapsules;
preferably, the mixed dispersion is granulated and dried by spray fluidized-bed drying to obtain microcapsules.

15. The preparation method according to claim 1, wherein,
in step (3), silicon dioxide and corn starch are mixed well and used as a coating material to perform spray fluidized-bed drying of the mixed dispersion obtained in step (2) in a spray dryer to obtain microcapsules.

16. The preparation method according to claim 15, wherein,
in step (3), silicon dioxide accounts for 0.1 to 1 parts by weight, and/or corn starch accounts for 5 to 25 parts by weight.

17. The preparation method according to claim 15, wherein, in step (3),
a spray tower inlet air temperature is controlled at 140° C. to 160° C., a fluidized-bed temperature is controlled at 40° C. to 45° C., and an atomizer frequency is at 24 to 26 Hz.

18. The preparation method according to claim 1, comprising the following steps:
1) SAMe is mixed with an appropriate amount of cyclodextrin, placed in a ball mill, ground at a speed of 100 to 350 r/min for 15 to 30 minutes, and passed through an 80 to 120 mesh sieve;
2) the product of step 1) is dispersed in purified water at 20° C. to 40° C.;
3) an appropriate amount of an acid-resistant filler is added and mixed well;
4) an appropriate amount of a protein wall material is dissolved in water, adjusted to have a pH value of 10 to 12 to obtain an alkaline protein aqueous solution, and then an appropriate amount of a prebiotic sugar is added, heated to 80° C. to 95° C., stirred for 15 to 30 minutes, and reacted under thorough stirring;
5) the product of step 3) and the product of step 4) are mixed, then subjected to ultrasonic treatment under negative pressure (the pressure is −0.07 MPa to −0.1 MPa) with an ultrasonic power of 240 to 480 w (preferably 300 to 400 w) and a treatment time of 6 to 8 minutes to obtain a uniform mixed dispersion, and a temperature kept at 20° C. to 40° C.;
6) an appropriate amount of silicon dioxide is added to corn starch and mixed well, and used as a coating material to perform spray fluidized-bed drying of the mixed dispersion of step 5) in a spray dryer with a spray tower inlet temperature controlled at 140° C. to 160° C., a fluidized-bed temperature controlled at 40° C. to 45° C., and an atomizer frequency at 24 to 26 Hz; after being sieved, SAMe microcapsules with a particle size between 40 and 70 mesh are obtained;
preferably, the preparation method comprises the following steps:
i) 50 to 90 parts by weight of SAMe is mixed with an appropriate amount of cyclodextrin, placed in a ball mill, ground at a speed of 100 to 350 r/min for 15 to 30 minutes, and passed through an 80 to 120 mesh sieve;
ii) the product of step i) is dispersed in purified water at 20° C. to 40° C.;
iii) 0.5 to 5 parts by weight of an acid-resistant filler is added and mixed well;
iv) 5 to 10 parts by weight of a protein wall material is dissolved in water, adjusted to have a pH value of 10 to 12 to obtain an alkaline protein aqueous solution, and then 0.5 to 5 parts by weight of a prebiotic sugar is added, heated to 80 to 95° C., stirred for 15 to 30 minutes, and reacted under thorough stirring;
v) the product of step iii) and the product of step iv) are mixed, then subjected to ultrasonic treatment under negative pressure (the pressure is −0.07 MPa to −0.1 MPa) with an ultrasonic power of 240 to 480 w (preferably 300 to 400 w) and a treatment time of 6 to 8 minutes to obtain a uniform mixed dispersion, and a temperature kept at 20° C. to 40° C.;
vi) an appropriate amount of silicon dioxide is added to corn starch and mixed well, and used as a coating material to perform spray fluidized-bed drying of the mixed dispersion of step v) in a spray dryer with a spray tower inlet temperature controlled at 140° C. to 160° C., a fluidized-bed temperature controlled at 40° C. 45° C., and an atomizer frequency at 24 to 26 Hz; after being sieved, SAMe microcapsules with a particle size between 40 and 70 mesh are obtained.

19. A SAMe microcapsule, which is obtained by the preparation method according to claim 1.

20. A composition, which comprises the SAMe microcapsule according to claim 19 and one or more pharmaceutically or bromatologically acceptable excipients;
preferably, the composition is a tablet, a soft capsule, a hard capsule, a powder, or a pill.
